(19) United States
(12) Patent Application Publication
(10) Pub. No.: US 2011/0027348 A1
(43) Pub. Date: Feb. 3, 2011

(54) COMPOSITION AND METHOD INHIBITING INFLAMMATION

(76) Inventor: Janos FEHER, Budapest (HU)

Correspondence Address: Pearl Cohen Zedek Latzer, LLP, 1500 Broadway, 12th Floor, New York, NY, 10036, US

(21) Appl. No.: 12/675,504

(22) PCT Filed: Aug. 27, 2007
(86) PCT No.: PCT/HU08/00098

§ 371 Date: Feb. 26, 2010

(30) Foreign Application Priority Data
Aug. 27, 2007 (HU) P0700552

(51) Int. Cl. A61K 9/127; A61K 9/107; A61K 35/74; A61K 36/064; A61K 36/062; A61P 9/10; A61P 9/06; A61P 25/28; A61P 29/00; A61P 27/02; A61P 25/00; A61P 11/00; A61P 3/04; C12N 5/00; C12N 1/20

(52) U.S. Cl. 424/450; 424/400; 424/780; 424/195.16; 435/404; 435/253.6

(57) ABSTRACT

Composition and method for preventing, treating or attenuating inflammatory diseases by way of inhibiting lipid peroxidation and subsequent elementary inflammation through ensuring the presence of the following biologically active ingredients in the PMRS of cells involved in inflammation: i) at least one killed probiotic and ii) at least one omega 3 FA and iii) vitamin E and iv) ubiquinone by introducing a composition comprising any of the missing ingredients.

This invention is related to compositions inhibiting inflammation and to methods for treating inflammation of humans and mammals with compositions according to the invention. By way of introducing their active principles into the plasma cellular membrane redox system (PMRS) these compositions inhibit generation of lipid peroxides from membrane lipids which represent the earliest event of the inflammatory chain or cascade. Thus, these compositions may be used to prevent, attenuate and/or inhibit inflammation and inflammation-related diseases. The basis of the invention is a method for preventing, treating or attenuating inflammatory diseases by way of inhibiting lipid peroxidation and subsequent elementary inflammation through ensuring the presence of the following biologically active ingredients in the PMRS of cells involved in inflammation:
1. at least one killed probiotic and
2. at least one omega 3 FA and
3. vitamin E and
4. ubiquinone

by introducing a composition comprising any of the missing ingredients.

Some terms used frequently in this specification are defined as follows (if not specifically stated otherwise):

Anaerobic glycolysis or Fermentation: a process of energy production in a cell under anaerobic conditions (with no oxygen required). Sugars are the common substrate of fermentation, and typical examples of fermentation products are lactic acid, and hydrogen.

Arachidonic acid (AA): omega 6 type PUFA, precursor of prostaglandines and leukotrienes.

Composition for Elementary Inhibition of Inflammation

Combination according to the present invention of compounds probiotics, omega 3 FA and vitamin E as each of them should come from exogeneous sources

Cyclooxygenase (COX): enzyme transforming AA into prostaglandines

Elementary Inflammation (definition of the inventor):

formation of lipid peroxides from membrane lipids, which represents the initial event in generating inflammation (“inflammatory cascade”). This is always associated with altered membrane functions unless specifically indicated.

Inflammation: is a reaction of human and animal organism to noxious agent or stimuli characterized by rubor (redness), color (heat), tumor (swelling), dolor (pain), function laesa (loss of function). Prostaglandines and leukotrienes are the principal proinflammatory cytokines responsible for inflammation.

Lipoxygenase (LOX): enzyme transforming AA into leukotrienes.

Metabolic antioxidant process: electrons from anaerobic glycolysis through NAD(P)H and NADH are transported into PMRS, and these electrons prevent generation of lipid peroxides as well as regenerate oxidized CoQ, vitamin E and omega 3 FA in this order.

Method for Elementary Inhibition of Inflammation: methods for inhibiting generation of lipid peroxides in the PM, (synonymously Inhibition of Elementary Inflammation); definition first used by the inventor

Mitochondrium a membrane-enclosed organelle, found in most eukaryotic cells, except red blood cells. They generate most of the cell's supply of ATP used as a source of chemical energy.

NOX (NADH oxidase): located at the outer side of the plasma membrane, transfers electrons from reduced CoQ to the molecules on the outer side of plasma membrane (ascorbat, proteins, oxygen)

Omega 6 fatty acid: a family of PUFA, which have in common, a carbon-carbon double bond in the ω-6 position

Omega-3 fatty acids: a family of PUFA, which have in common a carbon-carbon double bond in the ω-3 position

Organelles: submicroscopic structures of cell having specialized functions. These are mitochondria, endoplasmic reticulum, Golgy apparatus, lysosomes, and peroxisomes.

Phospholipase A2: located at the inner side of plasma membrane, activated by lipid peroxides, and causes release of arachidonic acid from membrane lipids.

Plasma Membrane Redox System (PMRS): an electron transport system composed of (i) NADH oxidase (NOX) (ii) ubiquinone (CoQ) (iii) Ubiquinone reductase.

Polyunsaturated Fatty Acids (PUFA): Fatty acids containing more than one carbon-carbon double bond.

Probiotic microorganisms or their derivates, which confer a beneficial health effect on the host organism. These are Lactobacilli, Bifidobacteria, Saccharomyces boulardi, Saccharomyces cerevisiae, Monascus pupurea.

Ubiquinone (CoQ): an intramembrane redoxactive molecule. Its reduced form is called ubiquinol; its oxidized form is called ubiquinone.

Ubiquinone reductase: located at the inner side of the plasma membrane it transfers electrons from NAD(P)H and NADH to CoQ.

Abbreviations used in this specification are explained as follows:

| | |
|---|---|
| AA | arachidonic acid [20:4 (n-6)] |
| ALA, | α-linolenic acid [18:3 (n-3)] |
| BB | Bifidobacterium |
| CoQ | ubiquinone, Coenzyme Q, |
| CoQ10 | ubiquinone 10, Coenzyme Q10, |
| DHA | docosahexaenoic acid [22:6 (n-3)] |
| EPA | eicosapentaenoic acid [20:5 (n-3)] |
| LA | linoleic acid [18:3(n-6] |
| LB | Lactobacillus or cell free (killed) extract of Lactobacillus |
| | in water (suspension or solution) |
| LTA | lipoteichoic acid |
| NAD(P)H) | Nicotinamide Adenine Dinucleotide Phosphate H |
| NADH | Nicotinamide Adenine Dinucleotide H |
| NOX | NADH oxydase |
| NSAID | Non-Steroid Anti-Inflammatory Drug |
| PM | Plasma Membrane |
| PMRS | Plasma Membrane Redox System |
| PUFA | PolyUnsaturated Fatty Acid |
| ROS | Reactive Oxygen Species (“free radicals”) |
| SAID | Steroid Anti-Inflammatory Drug |

It is well known that all human and animal cells are separated from their surroundings by a specially organized membrane, the so-called plasma membrane (PM). From a biochemical point of view, the basal structure of PM is a lipid bilayer, composed mostly of phospholipids, while cholesterol is present in minor quantities. Phospholipid molecules arranged in the PM so that the phosphoric acid “polar heads” face towards the membrane's internal and external surface while the fatty acid chains, “apolar tails”, face each other and thus form the membrane's internal hydrophobic region. Further component of PM are transmembrane protein, peripheral proteins, glicolipids and glycoproteins. The organelles found inside the cells have a similar membrane structure.

Membrane lipids are fundamentally responsible for the functioning of PM. The quantity and ratio of phospholipid-cholesterol, the unsaturated-saturated fatty acids, and the omega 3-omega 6 fatty acids are characteristics of PM functionality. It is generally accepted that the lipid composition of membranes effects their functioning in at least 6 different ways, namely they modify (i) the membrane's fluidity, (ii) the membrane's permeability (the functioning of the ion-channels), (iii) the activity of the enzymes connected to the membrane, (iv) the density and the affinity of the membrane receptors, (v) the release and activity of the neurotransmitters, and (vi) the release of the proinflammatory cytokines

In this aspect the polyunsaturated fatty acids (PUFA), first of all the members of omega 3 and omega 6 fatty acid (FA) families, have a particularly important role. Precursors of PUFA, alpha-linolenic acid, (ALA) (ω 3) or [18:3 (n-3)] and linoleic acid (LA) (ω 6) or [18:3 (n-6)] come exclusively from meals, since the human body does not have the enzymes necessary to produce them. Therefore the proper quantity and quality of PUFA intake in the daily diet is crucial to replenish even the physiological PUFA loss, thus to maintain the PM normal functioning. The PUFA loss gets worse with certain diseases, so the PUFA intake must be increased.

It is known that because of eating habits in the industrialized countries, today's diets do not contain the necessary omega 3 FA and the ratio of omega 6 is higher than optimal. A reduced omega 3 FA content in the cell membranes, respectively an altered omega 3-omega 6 ratio has been found in inflammatory and degenerative diseases and tumors, and in certain congenital diseases, so it has been linked to the development of these diseases. It has also been found, that addition of omega 3 FA, or of their natural precursors to the daily diet, lead to an increased omega 3 FA content in the cell membranes, a decreased occurrence of the above listed diseases and a better recovery. The recommended daily allowance for DHA+EPA amounts to 650 mg.

Vitamin E and the CoQ molecules are embedded into the membrane lipids. They have a very important biological role in the maintenance of the normal membrane structure and functioning. Vitamin E is known as a fat-soluble vitamin. There are 8 known forms of vitamin E: α-, β-, γ-, and δ-tocopherols contain saturated phytyl side chains and α-, β-, γ-, and δ d-tocotrienols have 3 double bonds in the side chain. The α-tocopherol molecule is the most potent. It is a 6-hydroxychroman derivative with methyl groups in position 2,5,7, and 8 and a phytyl side chain attached at carbon 2. The chroman ring is the redox-active part, while the phytyl side chain binds it to the PM. Due to its lipophilic nature, vitamin E accumulates in cellular membranes. The major function of vitamin E is to act as a natural antioxidant by scavenging free radicals and molecular oxygen. Vitamin E is important for preventing peroxidation of PUFA in membranes. It comes exclusively from the diet. The recommended daily allowance is 10 mg.

It is further known that CoQ is a fat soluble, redox-active molecule, that has two parts: (i) a quinon ring, which is able to pick-up and release 2 electrons, and (ii) a hydrophobic isopren side-chain connected to it, which locks the molecule in the hydrophobic zone of the PM. In humans, this side-chain has 10 subunits (this is what the 10 refers to in CoQ10). CoQ10 can be found in all cell membranes, that suggests an important biological role. It has been also found in the membranes of bacteria indicating that it had been developed early on during evolution and it has a fundamental role in the antioxidant defense mechanism. In a normal circumstance the body covers its CoQ10 needs with endogenous biosynthesis. This biochemical process is tightly connected to the cholesterol synthesis. In earlier studies, supplementing the daily diet with CoQ10 favorably influenced the inflammatory and degenerative diseases, the tumors and the course of some inherited diseases, too. Addition of omega 3 FA strengthened the effect of the CoQ10. This quality of CoQ10 has been credited exclusively to its effects on the mitochondrial metabolisms, more specifically to its effects on the electron transport chain, which is considered in its totality as an “anti-apoptotic” effect (Biochim Biophys Acta. 2004; 1660:171-199). The recommended daily allowance for CoQ10 is not determined.

Recent studies described a molecular organization denominated Plasma Membrane Redox System (PMRS) in the PM. A current concept assigned a central role to the CoQ10 in the functioning of this redox system. According to this concept CoQ10 takes electrons from the cytosolic NADH and NAD(P)H and gets reduced, and then transfers these electrons to the ascorbat or thiol-containing molecules reside on the external leaflet of the PM. Both steps of this transmembrane electron transport are catalized by specific enzymes (J. Exp. Biol. 2000; 2031513-1521).

Our concept extended this theory at two points assigning active role to the omega 3 FA and vitamin E of the PM and to the cytosolic anaerobic glycolysis.

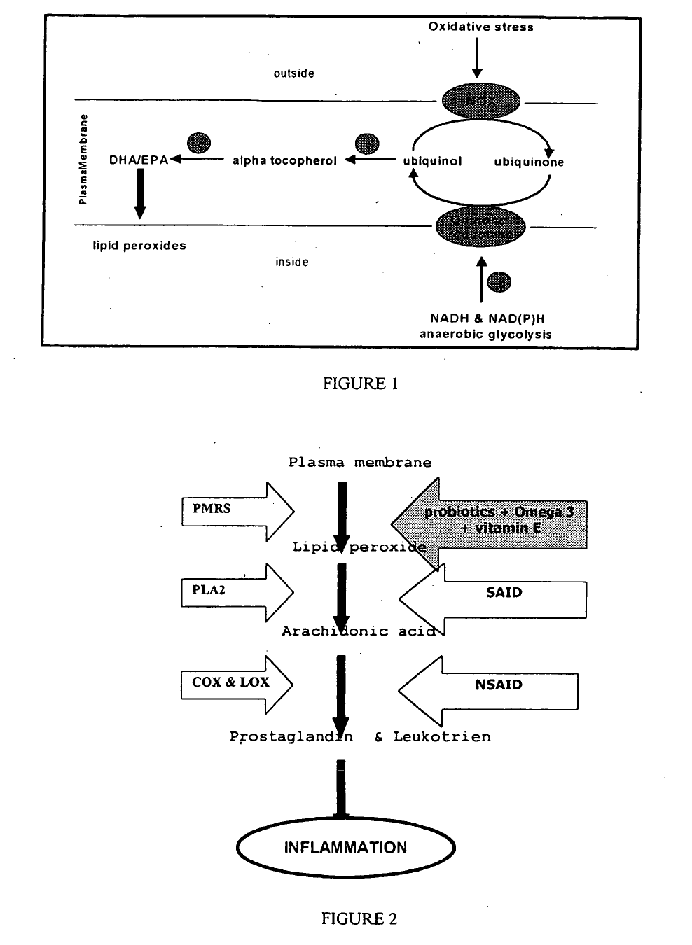

The flow chart represented in FIG. 1 schematically summarizes the molecular mechanism of PMRS according to our concept.

This flow-chart summarizes the following functions of the PMRS:
1. Transmembrane electron transport (orthodox function): delivery of electrons from cytosolic NADH and NAD(P)H to PM surface molecules (ascorbates, proteins, molecular oxygen),
2. Intramembrane electron transport (new function): delivery of electrons to the intramembrane alpha-tocopherol and omega 3 FA for maintaining or restoring adequate saturation of PM lipids,
3. Anaerobic glycolysis (new function) is the principal source of electrons for both transmembrane and intramembrane electron transports

An oxidative stress to membrane causes peroxidation of omega 3 FA. A loss of one or more double bounds results in conformational changes in fatty acid chains, which may have two consequences: (i) modification of the transmembrane functions, and (ii) initiation of inflammation. As these two functional changes are two consequences of the same processes, we introduced a new term “elementary inflammation”, which covers both aspects of membrane dysfunction

Ad (i) It is generally accepted that transmembrane proteins and peripheral proteins perform most of the membrane functions. Recent scientific findings showed that amino acid composition of membrane proteins and fatty acid composition of membrane lipids should be matched for normal functioning. Proper function of membrane proteins is conditioned by well-defined lipid environment (“lipid-shell”), and any change in lipid shell results in modification of membrane protein functions. (Biochim Biophys Acta. 2008; 1778:1545-75).

Ad (ii) The forming of lipid peroxides means a starting point for the beginning of inflammatory processes. The lipid peroxides activate the phospholipase A2 enzyme, which results in the release of arachidonic acid (AA) from the membrane phospholipids. From the AA, with the help of cyclooxigenase enzyme (COX) prostaglandines are formed, or with the help of lipoxigenase (LOX) leukotrienes are formed. Molecules with many and very diverse biological effects belong to both groups, including the pro-inflammatory molecules.

From the two main groups of anti-inflammatory medications used today, steroids inhibit the AA production with blocking of phospholipase A2, while non-steroids inhibit forming of prostaglandines and leukotrienes with the blocking of COX and LOX. Mechanisms and treatment possibilities including the present invention are summarized in the flow sheet of FIG. 2.

TABLE 1
EXPLANATION OF THE FIGURES

| Number | Title | Explanation |
|---|---|---|
| FIG. 1 | Plasma Membrane | Summarizes schematically the molecular |
| | Redox System | mechanism of Plasma Membrane |
| | | Redox System according to our concept. |
| FIG. 2 | Mechanism and | Summarizes schematically mechanisms |
| | Treatment of | and treatment possibilities using |
| | Inflammation | anti-inflammatory medications, including |
| | | the present invention. |

It is generally accepted that inflammation is the body's coordinated form of defense against the internal and external influences, and it has a key role in maintaining the homeostasis of the cells and organism.

In case of immunogenic inflammation, a form of white blood cells, the monocyta or macrophage or dendritic cells have an important role in the detection of the foreign substance and in the production of the so-called pro-inflammatory cytokines responsible for creating inflammation.

In case of neurogenic inflammation, the sensory nerves sense the foreign influences, which leads to the production of pro-inflammatory neuropeptides.

These two mechanisms always occur mixed together. This explains the well-known fact, that inflammation is always accompanied by pain, while pain can cause inflammation. Therefore the reduction of inflammation alleviates the pain, and the soothing of pain lessens the inflammation.

In most cases though, the inflammatory reaction can be so strong, that independently from the original reason, the inflammation itself causes damage to the body. Inflammation results in increased metabolism, which leads to the release of an extreme quantity of cell- and tissue-damaging reactive oxygen species (ROS). This explains why the reduction of inflammation goes together with the improvement of the diseased condition, and with the decrease of the damages.

Thanks to the findings obtained in the last decade, more and more data indicate the role of inflammation not merely in infections, allergies and (auto) immune diseases, but also in degenerative diseases characterized by cell loss (apoptosis) and in tumors characterized by cell proliferation where inflammation precedes or causes the degenerative disease or the tumor.

Some main groups of diseases where inflammation has been found to play a pathological role representing potential targets of treatment of the present invention:
Infections: in bacterial and viral infections, and in the inflammations of the mucus membranes that come with them, like f.e. conjunctivitis, gingivitis, bronchitis, gastritis, vaginitis, and cystitis.
Allergies, like f.e. bronchial asthma, dermatitis, allergic conjunctivitis, hay fever, etc.
Autoimmune diseases, like f.e. rheumatoid arthritis, juvenile (type 1) diabetes, Crohn's disease, ulcerative colitis, psoriasis, lupus erythematosus, sclerosis multiplex. etc.
Age related degenerative diseases like f.e. Alzheimer's, dementia, Parkinson's disease, amyotropic lateral sclerosis, age related macular degeneration, glaucoma, cataract, otosclerosis, osteoporosis, arthrosis, muscle atrophy, hair loss (baldness), etc.
Neuropsychiatric diseases, like f.e. schizophrenia, depression, anxiety, panic disorder, etc
Metabolic syndrome and related diseases, that includes obesity, type II diabetes, high blood pressure, high blood fat, and fatty liver, atheriosclerosis and its consequences like f.e. coronary disease, stroke, etc.
Neovascular diseases, diabetic retinopathy, wet type AMD, proliferative retinopathy of premature infants.
Soft tissue damages, like trying sport performances (marathon), sport injuries, contusions, large burns and frost-bites, post-operative inflammations, etc.
Tumors, like f.e. benign and malign tumors of the prostate, and of the cervix, breast cancer, lung cancer, intestinal cancer, lymphoma, etc.

There are several drawbacks of the currently used SAID and NSAID treatments. Both groups have serious side effects that occasionally can be fatal. Furthermore, neither of them influences significantly the natural course of degenerative diseases and tumors. It is also well known that omega 3 FA have a slight anti-inflammatory effect through modifying generation of pro-inflammatory cytokines. Thus they act in the advanced phase of the inflammation cascade. Substances causing the end of the inflammation also result from omega 3 FA.

Numerous publications in the international literature, widely used compositions in clinical practice, and several patents described the biological and pharmacological influence of omega 3 FA, vitamin E and CoQ either separately or in combination. Here we mention some of them.

It is known that a combination of omega 3 FA, vitamin E and CoQ has been suggested to treat dysfunctions of mitochondrial metabolism, called mitochondriopathies (EP 1123093). This was based on an observation that omega 3 FA enhances incorporation of CoQ into mitochondrial membranes, where CoQ plays an essential role in the mitochondrial metabolism. Similarly: improvement of mitochondrial metabolism was suggested by a combination containing omega 3 FA, vitamin E, CoQ and L-carnitine or acetyl-l-carnitine (European application 05014812.1).

According to a human dietary study vitamin E and CoQ increased EPA plasma levels, also when fish oil was added to the diet. In some persons decreased AA levels were found. These authors supposed that it makes less favourable conditions for generation of cytokines and thus may have NSAID-like effects (J. of Nutritional & Envir. Med., 1998; 8:25-34).

There is also known a food supplement containing omega 3 FA and CoQ with carotenoids, bioflavonoids, water-soluble vitamins and metal ions (U.S. Pat. No. 6,579,544).

It is also known that lipid emulsions containing soy oil and/or fish oil may be used for enteral or parenteral nutrition for critically ill patients. When fish oil was added to the composition the nutritive effects were accompanied with some decrease of inflammation (Proc. Nutr. Soc., 2006, 264-277).

For treating dysfunction of vascular endothelium some food supplements containing combinations of omega 3 FA, CoQ and sugar-amines, such as glucosamine, galactosamine, were suggested in tablets or powder (U.S. Pat. No. 6,930,099

The above known combinations also have the significant drawback that the incorporation of these substances is a spontaneous and slow process. Thus their therapeutic effect is casual or low and it develops slowly.

Probiotics—as defined by the Food and Agricultural Organization (FAO) of the United Nations—are “live microorganisms administered in adequate amounts which confer a beneficial health effect on the host.” Earlier, probiotics have been used orally as foods, or food supplements, or locally and their physiological benefits were attributed to their immuno-modulating effects such as inhibiting colonization of pathogens on the gastrointestinal or uro-genital mucous membranes, and enhancing phagocytic activity of monocytes, macrophages and dendritic cells, stimulating T cell differentiation, and enhancing secretion of immunoglobulin A (IgA). (WO 01/37862 A3 and USP 20080175685 A1 Jul. 24, 2008). This immuno-modulating effects of both live and killed probiotics were attributed to their cell-wall component, lipoteichoic acid (LTA), an endotoxin of Gram positive bacteria (Proc Natl Acad Sci USA. 2005; 102(29):

### BRIEF SUMMARY OF THE INVENTION

It is an object of the present invention to provide compositions for preventing, treating or attenuating inflammatory diseases by the use of (i) cell free extract of killed probiotics, or (ii) a combination of cell-free extract of killed probiotics and omega 3 FA and vitamin E, or (iii) a combination of cell-free extract of killed probiotics and omega 3 FA and vitamin E and further pharmacologically acceptable active substances.

It is another object of the invention to provide methods for preventing, treating or attenuating inflammatory diseases by the way inhibiting lipid peroxidation and subsequent elementary inflammation through introducing the active ingredients of these compositions into the PMRS of the targeted cells.

Yet it is another object of the invention to provide methods for preventing, treating or attenuating inflammatory diseases by the use of above compositions in lipid emulsion or in water solution formulated for enteral, parenteral and topical use.

It is a further object of the invention to provide the use of the above compositions and methods for preventing, treating or attenuating inflammation related to infectious, immune, degenerative and neoplastic diseases.

### DETAILED DESCRIPTION OF THE INVENTION

One scientific basis of our invention is our original observation according to which the PMRS, the surrounding omega 3 FA and vitamin E, as well as the cytoplasmic anaerobic glycolysis, form a functional unit, which plays an essential role for survival and adaptation of human and animal cells in both normal conditions and in diseases. Dysfunctions of this unit manifest as elementary inflammation, which is the earliest event of inflammation and subsequent inflammatory diseases. Thus, the PMRS represent a new therapeutic target for preventing, treating or attenuating inflammation and inflammation related diseases. The present invention describes compositions and methods suitable to reach these objectives.

The introduction of probiotics in the treatment regime of inflammation and related diseases represent an original approach. It comes from our observation that exogenous probiotics stimulate the anaerobic glycolysis of host cells, which produce more NADH and NAD(P)H to release more electrons into the PMRS for maintaining or restoring adequate redox state of PMRS. This metabolic antioxidant/anti-inflammatory effect comes from the cytoplasmic fraction of probiotics. It is well known that probiotics are internalized by phagocytes (macrophages, monocytes, endothelial cells, microglia), which are primarily involved in the inflammation. In this way genetic material (DNA, RNA) encoding the anaerobic glycolysis is transported into the host cells, and incorporated into the host genome enhancing their own anaerobic metabolism. This horizontal gene transfer is well know in gene-research. It should be kept in mind that genes encoding anaerobic glycolysis are highly conserved during the evolution, thus these genes from probiotics are compatible with those of the host cells. In this context, introduction a cytoplasmic extract of probiotics into human and animal organism works as a “metabogenic vaccine” which stimulate anaerobic metabolism, in contrast to the currently used “immunogenic vaccines” which use substances for generating immune responses.

Neither of the previous patents or scientific works mentioned that anaerobic metabolism of probiotics might be responsible for any beneficial effects of them either in connection with their immunostimulant effects or independently from those. Furthermore, no prior art described probiotic effects on elementary inflammation or on one of the features of elementary inflammation either in animal or human organism. Consequently, these properties of probiotics have never been applied in any treatment regime. Thus, the discovery that anaerobic metabolism of probiotics prevents elementary inflammation is certainly a novelty and it is a key scientific support to this invention.

The primary embodiment of this invention is a composition for elementary inhibition of inflammation in human or mammal organism by inhibition of lipid peroxidation through introducing the following compounds into the cells involved in the inflammation,
a) an ingredient for elementary inhibition of inflammation:

extract of killed probiotics, and
b) pharmaceutically acceptable excipients, vehicles, preservatives and colorants.

In the composition the killed probiotic may be full or partial extract of Lactobacillus, and/or Bifidobacterium, and/or Saccharomyces cerevisiae or the combination thereof, and/or full or partial extract of biologically acceptable anaerobic bacteria.

In the composition the active ingredients may be the cytoplasmic fraction of killed probiotics, or the nucleotide components (DNA, RNA) of probiotics, or combination thereof, or these ingredients may also derived from the genetic modification or from the full or partial synthesis of probiotics' DNA and/or RNA

Preferred probiotics are killed Lactobacillus acidophilus, L. casei, L. plantarum, L. reuteri, L. rhamnosus, L. GG, L. bulgaricus, L. bifidus, L. caucasicus, L. brevis, L. cellobiosus, L. crispatus, L. curvatus, L. fermentum, L. gasseri, L. johnsonii, L. salivarus; and/or Bifidobacterium animalis subsp. lactis, B. bifidum, B. breve, B. infantis, B. longum, B. adolescentis, B. animalis, B. thermophilum, B. lactis, and/or Lactococcus lactis (formerly known as Streptococcus lactis), Streptococcus thermophilus, Bacillus coagulans, and/or Enterococcus faecalis, Enterococcus faecium, and/or Saccharomyces boulardii, Saccharomyces cerevisiae, Monascus purpureus

Favorably the quantity of probiotics is 0.01-1000 mg/dose, most preferably 0.1-10 mg/dose. This quantity was determined by the protein content of probiotic suspension.

This new invention is supported by another original observations that addition of omega 3 FA and vitamin E to probiotics surprisingly enhances the anti-inflammatory effects. This synergy comes primarily from the restoration of PM's omega 3 FA and vitamin E composition and less importantly from the simple addition of anti-inflammatory effects of probiotics and omega 3 FA. We have mentioned before that PM may contain low levels of these substances duo to either low dietary uptake or chronic environmental influences (smoking, toxins). In these conditions, addition of omega 3 FA to probiotics is essential for obtaining adequate therapeutic effects. No prior art is known on this synergy of probiotics, omega 3 FA and vitamin E.

A further embodiment of this invention is a composition for elementary inhibition of inflammation in human or mammal organism by inhibition of lipid peroxidation through introducing the following compounds into the cells involved in the inflammation,
a) combination of ingredients for elementary inhibition of inflammation:

extract of killed probiotics, and

omega 3 FA and

vitamin E, and
b) pharmaceutically acceptable excipients, vehicles, preservatives and colorants.

The composition according to the invention may comprise the omega 3 FA in the form of pharmacologically identical natural form or source of ALA, and/or EPA and/or DHA, and/or ester thereof, preferable an ethyl-ester or trigliceride, and/or omega 3 containing phospholipids preferably phosphatidylinositol, phosphatidylcholine phosphatidylethanolamine, phosphatidylserine, and sphingomyelin or combinations thereof. The vitamin E may be pure α-tocopherol and/or β-, γ-, or δ-tocopherol, α-, β-, γ-, or δ-tocotrienol and/or their natural, semi-synthetic or synthetic esters.

In the composition the omega 3 FA may be present in form of its precursor such as fish oil. This may be prepared from any part of see-fish preferably from salmon, cod-liver. Also vegetable oil preferably from linseed, rape seed, grape pips and/or the oil derived directly from micro-algae or any other marine living organisms can be used.

Favourably the quantity of omega 3 FA or their ester is 100-1500 mg/dose, most preferably 250-750 mg/dose.

Favorably the quantity of vitamin E or its ester is 5-500 mg/dose, most preferably 15-100 mg/dose.

It was also mentioned, that ubiquinone is synthesized endogeneously to cover the body's need. However, in certain conditions it may be insufficient (for example, in use of cholesterol lowering statins) and exogeneous supplement is needed for improving functions of PMRS.

A further embodiment of this invention is a composition for elementary inhibition of inflammation in human or mammal organism by inhibition of lipid peroxidation through introducing the following compounds into the cells involved in the inflammation,
a) combination of ingredients for elementary inhibition of inflammation:

extract of killed probiotics, and

omega 3 FA and

vitamin E, and

ubiquinone, and
b) pharmaceutically acceptable excipients, vehicles, preservatives and colorants.

Favorably the quantity of ubiquinone or its water-soluble derivates is 10-500 mg/dose, most preferably 20-100 mg/dose.

Compositions according to this invention may further contain pharmacologically compatible ingredients such as vitamin A, B, C, D, F, K and/or corticosteroids, sex-steroids, metal ions (sodium, calcium, magnesium, potassium, phosphor, zinc, iron, selenium) and L-carnitine, aminocarnitines, alpha-lipoic acid, glutathion, essential amino acids, bioflavonoids, polyphenols, terpenes, alkaloides (berberine), volatile oils, amino acids, antibiotics, glycosamino-glycans (hyaluronic acid, chondroitin-sulphate, heparin, heparin-sulphate).

The compositions may further contain formulation additives such as excipients, vehicles, preservatives and colorants selected according to the actual ingredients and the intended method of administration.

According to our own studies a formulation for lipid emulsion (oil in water or water in oil) significantly increased the efficacy of our combinations. The vascular endothelial cells and white blood cells may directly incorporate active substances by receptor mediated processes and/or phagocytosis. Both play an essential role in generating inflammation. Furthermore, in this way a lower dosage may be effective. Finally, in the clinical practice, by parenteral lipid emulsion, we may avoid all drawbacks of enteral administration, first of all malabsorption of lipids typical and common in biliary dysfunctions. Topical administration of lipid emulsion in form of solution, gel or ointments also increases the bioavailability of these compounds. They can reach directly the involved inflammatory cells on the conjunctiva, on the gastrointestinal or urogenital mucous membranes or on the skin surface.

According to our invention, lipid emulsion similarly to other known and used compositions is a mixture of water (with or without additional water-soluble substances) and oil (with liposuluble substances) and they form a stable emulsion specifically in the presence of a suitable emulsifier. Our invention comprises both “water in oil” and “oil in water” types depending on the mass ratio of water and oil and the intended use form.

A further and particularly important embodiment of our invention is the formulation of lipid emulsion for delivery of the active ingredients. These lipid emulsions contain
A combination of the above listed ingredients
Water forming a stable lipid emulsion in which the mass ratio of omega 3 FA or their esters to water is 100:1-100000; and
A pharmacologically acceptable emulsifier.
   In this context probiotics, omega 3 FA, and vitamin E represent all the previously listed forms ingredients in which these active ingredients are present (precursors or derivates) without repeating them again.

Emulsifiers in lipid emulsions favorably are either of egg- or soy-lecithin, bile, bile acids, Tween® MT, polyvinyl alcohol.

The particle size in lipid emulsion may be 0.001 to 10 micros, preferable 0.01 to 5 micron.

Further ingredients/excipients of lipid emulsions (conservants, pH and osmolarity stabilizers) are identical with those of the commercially available products, but the quality of parenteral administration should be guaranteed (see U.S. Pat. No. 5,760,020)

According to our invention lipid emulsions are particularly favorable for parenteral (intravenous, intramuscular, intradermal, intraarticular, intraocular, intralesional, para-lesional, subcutaneous) application. In these cases the lipid emulsion may contain further specific pharmacologically acceptable excipients. This formulation is very suitable for rapid delivery of active ingredients for reaching prompt effects on the sites of inflammation. In certain cases this speed may be life saving.

Lipid emulsions may also be formulated for enteral (oral, rectal) transdermal, and nasal administration. In these cases they may contain further, pharmacologically compatible substances, excipients habitually used for similar formulations. The oral administration is particularly important.

For the aqueous phase of lipid emulsion either distilled water or a physiological salt solution is used most frequently.

Further possible formulations of lipid emulsion may be for direct administration in topical medication, such as eye-drops, gel, spray, ointment, solutions for lotion, which contain further known and pharmacologically and chemically compatible auxiliary substances and vehicles

The lipid emulsion may also be formulated for oral use in soft capsules. This composition—for enteral use—may contain all the previously listed preferred substances. Particularly favorably are compositions containing full extract of killed lactobacillus or that of a saccharomyces, omega 3 FA and vitamin E.

Another embodiment of this invention is a water-soluble composition for preventing or inhibiting elementary inflammation. These contain as active ingredients either of the following combinations:
A water-soluble full or partial cell-free extract of at least one of the probiotics.
A water-soluble salt or ester of at least one of the omega 3 FA, and water-soluble ester of natural, semi-synthetic or synthetic vitamin E,
Optionally a water soluble salt or derivate of the above listed ingredients
A pharmacologically acceptable water solution.
   In this context probiotics, omega 3 FA, vitamin E and the optional water soluble salt or derivate of the above listed ingredients represent all the previously listed forms ingredients in which these active ingredients are present (precursors or derivates) without repeating them again.

Such compositions may further contain water-soluble excipients, vehicles, preservatives and colorants.

According to our invention a water-soluble composition as described above preferably for parenteral use (via endovenous infusion or injection, subcutaneous or intramuscular injection); and for enteral use (via oral, intra-gastric, transrectal); and for topical use (lotions, eye-drops, nasal-drops, ear-drops, spray, cream, gel) and for liposome encapsulated delivery.

Compositions and combinations according to our invention are for both human and veterinary use either in enteral or parenteral or topical forms of administration.

Further embodiment of this invention is the use of above described compositions in lipid emulsion or in water-solution for preventing, treating and attenuating the following diseases:
Inflammation and sepsis, particularly those which are accompanied with bacterial, viral and fungal infections
Inflammation of mucous membranes such as conjunctivitis, periodotitis, oesophagitis, reflux disease, gastritis, enteritis, colitis, cholecystitis, cystitis, pyelo-nephritis, sinusitis, bronchitis, vaginitis, prostatitis, and related diseases thereof such as hepatitis, cirrhosis, nephritis, pleuritis, fibrosis cystica;
Autoimmune inflammation, such as rheumatoid arthritis, juvenile (type I) diabetes, Chron's disease, colitis ulcerosa, psoriasis, lupus erithematous, multipex sclerosis.
Allergic inflammation such as asthma, atopic dermatitis, hay-fever, allergic conjunctivitis, allergic rhinitis,
Neuropsychiatric diseases, such as schizophrenia, depression, anxiety, panic-disease.
Metabolic syndrome (dyslipidemia, hypertonia, diabetes, obesity, fatty liver).
Neovascular proliferative diseases, such as proliferative retinopathy, retinopathy of prematurity (ROP), malignant tumors.
Arterial hypertension and atherosclerosis and related diseases such as coronary heart disease, cardiac arrhythmia, chronic heart failure, nephrosis syndrome, ischemia-reperfusion, peripheral vascular diseases, acute or chronic cerebral ischemia, stroke.
Age-related degenerative diseases such as Alzheimer's, diseases, Parkinson's disease, amyotropic lateral sclerosis, age-related macular degeneration, glaucoma, cataract, otosclerosis, osteoporosis, osteoarthritis, sarcopenia, hairlessness (baldness), age-related skin changes.
Inflammation caused by soft tissue damage, such as postoperative inflammation, sport injuries, exhaustive sport activity (marathon running), contusions, burning, frost-bites
Inflammation related to neoplasia, such as prostate and uterus benign or malign tumors, breast cancer, lung cancer, colon cancer, and lymphomas, as well as for treating chemotherapy associated inflammations.
Inflammation evoked by common vaccines, such as influenza (flu), hepatitis, BCG, poliomyelitis, Di-Per-Te (diphtheria-pertussis-tetanus), epidemic parotitis, measles and anti-allergic vaccines.
Eye diseases, such as uveitis, diabetic retinopathy, age-related macular degeneration, glaucoma, cataract.

The present invention also relates to the preparation of the compositions according to the invention for treating the above-disclosed diseases.

A further embodiment of this invention is the use of compositions as described here for preparing an adjuvant to
Gene-transfer, natural or semi-synthetic or synthetic DNA o RNA transfers.
Culture medium either for cell-cultures, or tissue-cultures or bacterial cultures
Support survival of transplanted stem cells and organs.
   In these cases reduction of lipid peroxide generation and a metabolic support is achieved.

Our invention is illustrated in details by the following examples, without the intention to restrict our claims to these examples.

### Examples

### I. Biological Examples

Remark: In some of the examples of our priority document we used the term “LTD” instead of the now used “LB” for the applied compound: cell free (killed) extract of Lactobacillus in water. We presumed that LTD (lipoteichoic acid) was the active part of the actually added LB but this is not certain.

### Example I/1

### Influences of Omega 3 Fatty Acids, Vitamin E and CoQ10 on Immunogenic Inflammation

The purpose of this experimental model was to measure inhibitory influence of omega 3 fatty acids, vitamin E and CoQ10 separately and in combination, using methods disclosed in literature (J. Surg Res. 2002 September; 107(1):135-139). For evoking inflammation bacterial endotoxin (LPS) was used and changes in levels of a proinflammatory cytokin, TNF-alpha were measured in a monocyte/macrophage (RAW 264,7) culture. Results are summarized in Table 2. It is clear that both combinations show inhibition of inflammation, the 3 member combination a considerably higher one. Both combinations also show synergy being more effective than the sum of the compound effects. Both combinations were more effective than aspirin.

TABLE 2
Decrease in proinflammatory cytokine levels in immunogenic
inflammation

| | | | | Omega 3 + | |
|---|---|---|---|---|---|
| | | | | vitamin E + | |
| | | | Omega 3 + | CoQ10 | |
| cytokine | control | CoQ10 | vitamin E | (Example II/14) | aspirin |
| TNF- | 0% | −3% | −38% | −58% | −21% |
| alpha | | | | | |

### Example I/2

### Influences of Omega 3 Fatty Acids, Vitamin E and CoQ10 on Neurogenic Inflammation

Neurogenic inflammation was evoked in newborn (28 days) Sprague Dawley rats by intraperitoneal administration of capsaicin (50 mg/kg of body mass) as described in the literature (Acta Physiol. Hung. 1987; 69(3-4):323-32). Twenty-four hours before capsaicin injection 10 ml intravenous infusions each of CoQ10, or omega 3+vitamin E, or omega 3+vitamin E+CoQ10 were applied. Each of these combinations contained 1.0 g omega 3, 10 mg vitamin E and 10 mg CoQ10. Intravenous administration of both combinations containing either 2 or 3 compounds modified significantly both acute and chronic effects of capsaicin, as demonstrated on Table 3. Hypotensive shock and subsequent death were prevented by pre-treatment with omega 3+vitamin E in 26% and in the omega 3+vitamin E+CoQ10 in 64%, while in the control group only 22% survived. These findings strongly suggest that these pre-treatments reduced the release of proinflammatory neuropeptides, thus inhibited inflammation. Six weeks after the capsaicin injection, corneal transparency and sensitivity returned to normal in the pre-treated groups, while in the control group reduced transparency and sensitivity were observed. These observations strongly suggest that pre-treatment with the omega 3+vitamin E and CoQ10 combination either protected sensory nerves from capsaicin toxicity and/or supported the regeneration of damaged nerve fibers. If the omega 3+vitamin E and CoQ10 pre-treatments were separately applied the sum of these protective influences would be significantly lower than those of the composition containing all three compounds.

TABLE 3
Decrease in acute and chronic neurogenic inflammations

| | | | | Omega 3 + |
|---|---|---|---|---|
| | | | | vitamin E + |
| Neurogenic | | | Omega 3 + | CoQ10 |
| inflammation | control | CoQ10 | vitamin E | Example II/1 |
| Acute features | 22% | 22% | 26% | 64% |
| Chronic features | 20% | 25% | 45% | 100% |

### Example I/3

### Influences of Omega 3 Fatty Acids, Vitamin E, CoQ10 and LB on Endotoxin Induced Uveitis in Vivo

Experimental uveitis was evoked by administration of bacterial endotoxin into footpad of rats as described in the literature (J. Neurophysiol. 2005: 3815-3825.). Immediately after endotoxin administration, 10 ml intravenous infusion of omega 3+vitamin E+CoQ10, or omega 3+vitamin E+cell-free extract of lactobacillus (LB) or omega 3+vitamin E+CoQ10+LB were applied respectively while controls received physiological salt solution. This experiment was repeated and the same compositions were applied 7 days before endotoxin administration. Each of these combinations contained 1.0 g omega 3, 10 mg vitamin E and 10 mg CoQ10 and 0.01 mg LB. Symptoms of uveitis (hyperaemia, corneal oedema, state of the humour aqueous) were evaluated and compared to controls as described in the cited publication. (Table 4). In both experiments inflammatory parameters were 60% lower in average in the treated groups as compared to the control one. These observations further suggest that the omega 3+vitamin E+CoQ10 combination was more effective in post-treatment, while the omega 3+vitamin E+LB in the pre-treatment.

TABLE 4
Influence of lipid emulsion on endotoxin
induced uveitis (EIU)

| | | Omega 3 + | Omega 3 + | Omega 3 + |
|---|---|---|---|---|
| | | vitamin E + | Vitamin E + | vitamin E + |
| | | CoQ10 | LB | CoQ10 + LB |
| EIU | control | (Example II/1) | (Example II/2) | (Example II/3) |
| Post- | 0% | 63% | 55% | 58% |
| treatment | | | | |
| Pre- | 0% | 51% | 59% | 64% |
| treatment | | | | |

### Example I/4

### Influences of Omega 3 Fatty Acids, Vitamin E, CoQ10 and LB on Experimental Sepsis

Experimental sepsis of rats was evoked by intraperitoveal administration of bacterial endotoxin as described in the literature (Crit. Car. 2006; 10(4):R124). Immediately after endotoxin administration 10 ml intravenous infusion of omega 3+vitamin E+CoQ10, or omega 3+vitamin E+cell-free extract of killed lactobacillus (LB) or omega 3+vitamin E+CoQ10+LB were applied, while controls received a commercially available lipid emulsion. This experiment was repeated and the same compositions were applied 7 days before endotoxin administration. Each of these combinations contained 1.0 g omega 3, 10 mg vitamin E and 10 mg CoQ10 and 0.01 mg LB. While the 72 hour survival was 0% in the control group, it was 75% in average in the treated groups. These observations further suggested that the omega 3+vitamin E+CoQ10 combination was more effective in post-treatment, while the omega 3+vitamin E+LB in the pre-treatment. The results are shown in Table 5.

TABLE 5
Influence of lipid emulsions on experimental sepsis

| | | Omega 3 + | | Omega 3 + |
|---|---|---|---|---|
| | | vitamin E + | Omega 3 + | vitamin E + |
| | | CoQ10 | vitamin E + LB | CoQ10 + LB |
| Survival | control | (Example II/1) | (Example II/2) | (Example II/3) |
| Post- | 0% | 79% | 65% | 68% |
| treatment | | | | |
| Pre- | 0% | 72% | 79% | 85% |
| treatment | | | | |

### Example I/5

### Influence of Omega 3 Fatty Acids, Vitamin E, CoQ10 and LB on Ischemia-Reperfusion in Vivo

Using well defined experimental conditions an acute cardiac arrest of dogs was evoked by injecting physiological salt solution into the pericardial sac (pericardial tamponade) for 3 to 5 minutes as earlier described by us (Ann Ophthalmol. 1979 June; 11(6):909-13.). Immediately after resuscitation intravenous lipid infusion was applied containing either 10 g omega 3 fatty acids+100 mg vitamin E+100 mg CoQ10 per 100 ml, or 10 g omega 3+100 mg vitamin E+0.1 mg LB per 100 ml. The final dosage was referred to 1 g omega 3/kg/24 hours. Controls received the same amount of physiological salt solution. From sacrificed animals small samples of the retina, heart muscle and skeletal muscle were studied with light and transmission electron microscopy. Special attention was paid to the plasma membrane alterations, and morphometric methods were applied for quantification of abnormalities as compared to controls. Results are shown in Table 6. These findings show that this treatment significantly decreased tissue damage caused by 3 minutes ischemia-reperfusion. The most prominent effects were found in the neuronal cells (retina) and heart muscle, less evident in skeletal muscle. Similar effects of treatment were found after 5 minutes ischemia-reperfusion, but the magnitude of efficacy was estimated about 15% less as compared to those of 3 minutes experiment. There were no significant differences between these two combinations, at least in this experimental model.

TABLE 6
Influence of omega 3, vitamin E, CoQ10 and LB on membrane
damage in ischemia-reperfusion

| | | omega 3 + | omega 3 + |
|---|---|---|---|
| | | E vitamin + | E vitamin + |
| | | CoQ10 | LB |
| Membrane damage | control | (example II/1) | (example II/2) |
| Retinal ganglion cell | 0% | 43% | 45% |
| Heart muscle | 0% | 23% | 19% |
| Skeletal muscle | 0% | 10% | 8% |

TABLE 7
In vitro growth and differentiation of RPE cells influenced
by omega 3 fatty acids, Vitamin E, CoQ10 and LB.

| | | | | | omega 3 + | omega 3 + | |
|---|---|---|---|---|---|---|---|
| | | | | | E vitamin + | E vitamin + | |
| | | | | | CoQ10 | LB | omega 3 + E vitamin + |
| | | | | | (example | (example | CoQ10 + LB |
| RPE | control | CoQ10 | Omega 3 | LB | II/3) | II/14 | (example II/15) |
| growth | 0% | 1% | 23% | 7% | 43% | 69% | 72% |
| differentiation | 0% | 3% | 21 | 4% | 38% | 71% | 67% |

### Example I/6

### Influences of Omega 3 Fatty Acids, Vitamin E, CoQ10 and LB on Retinal Cell-Culture

The aim of this study was to reveal the influence of omega 3 fatty acids, Vitamin E, CoQ10 and LB separately or in combination on the development and differentiation of retinal cells in culture (in vitro). Retinas of newborn (1-2 days) Wistar rats were isolated. and cultured as described in our previous paper (Int. J. Dev. Neurosc. 1998 August; 16(5):423-32). In six different combinations, the culture medium contained 10 g omega 3 fatty acids, 100 mg vitamin E, 100 mg CoQ10 and 0.05 microgram LB per 100 ml. At the 7 day stage size and differentiation of retinal pigment epithelial (RPE) cells were determined by morphometric analysis, as described in the cited work. Our results are presented in Table 7. These in vitro data show that (1) omega 3+vitamin E+CoQ10 enhanced both growth and differentiation of RPE cells; (2) this effect was higher than the sum of the effects of each of these compounds; (3) addition of LB further improved both cell-growth and differentiation; (4) the same results were obtained without CoQ10 in the culture medium, suggesting probably an enhanced endogen biosynthesis of CoQ10 by LB.

### Example I/7

### Influences of Cell-Free Extract of LB Omega 3 Fatty Acids and Vitamin E on Endotoxin Induced Uveitis in Vivo

Experimental uveitis was evoked by administration of bacterial endotoxin into footpad of rats as described in the literature (J. Neurophysiol. 2005: 3815-3825.). Immediately after endotoxin administration, 10 ml intravenous infusion of omega 3+vitamin E, or cell-free extract of LB+omega 3+vitamin E were applied respectively while controls received physiological salt solution. This experiment was repeated and the same compositions were applied 7 days before endotoxin administration. Each of these combinations contained 1.0 g omega 3, 10 mg vitamin E and 10 mg LB. Symptoms of uveitis (hyperaemia, corneal oedema, state of the humour aqueous) were evaluated and compared to controls as described in the cited publication. (Table 4). In both experiments inflammatory parameters were 60% lower in average in the treated groups as compared to the control one. These observations further suggest that the LB was more effective in post-treatment, while the LB+omega 3+vitamin E in the pre-treatment. A omega 3+vitamin E showed weak effect in both pre and post-treatment (* not formulated for Examples)

TABLE 8

| | | | | LB + |
|---|---|---|---|---|
| | | | | Omega 3 + |
| | | LB | | vitamin E |
| | | (Example | Omega 3 + | (Example |
| EIU | control | II/1) | vitamin E* | II/2) |
| Post-treatment | 0% | 63% | 25% | 58% |
| Pre-treatment | 0% | 59% | 11% | 64% |

### Example I/8

### Influence of LB, Omega 3 Fatty Acids and Vitamin E on Ischemia-Reperfusion in Vivo

Using well defined experimental conditions an acute cardiac arrest of dogs was evoked by injecting physiological salt solution into the pericardial sac (pericardial tamponade) for 3 to 5 minutes as earlier described by us (Ann Ophthalmol. 1979; 11:909-13.). Immediately after resuscitation intravenous lipid infusion was applied containing either 10 mg LB or 10 g omega 3 fatty acids+100 mg vitamin E per 100 ml, or 10 mg LB+10 g omega 3+100 mg vitamin E per 100 ml. The final dosage was referred to 1 g omega 3/kg/24 hours. Controls received the same amount of physiological salt solution. From sacrificed animals small samples of the retina, heart muscle and skeletal muscle were studied with light and transmission electron microscopy. Special attention was paid to the plasma membrane alterations, and morphometric methods were applied for quantification of abnormalities as compared to controls. Results are shown in Table 6. These findings show that this treatment significantly decreased tissue damage caused by 3 minutes ischemia-reperfusion. The most prominent effects were found in the neuronal cells (retina) and heart muscle, less evident in skeletal muscle. Similar effects of treatment were found after 5 minutes ischemia-reperfusion, but the magnitude of efficacy was estimated about 15% less as compared to those of 3 minutes experiment. There were no significant differences between these two combinations, at least in this experimental model. Neither LB alone nor a combination of omega 3+vitamin E showed weak effect in both pre and post-treatment (* not formulated for Examples)

TABLE 9

| | | | | LB + |
|---|---|---|---|---|
| | | | | omega 3 + |
| | | LB | omega 3 + E | E vitamin |
| Membrane | | (Example | vitamin | (example |
| damage | control | II/1) | (*) | II/2) |
| Retinal | 0% | 10% | 13% | 45% |
| ganglion | | | | |
| cell | | | | |
| Heart | 0% | 8% | 15% | 19% |
| muscle | | | | |
| Skeletal | 0% | 7% | 10% | 18% |
| muscle | | | | |

### Example I/9

### Influence of LB, Omega 3 Fatty Acids and Vitamin B on Metabolic Syndrome

Metabolic Syndrome is a cluster of diseases characterized by (i) Impaired lipid metabolism (high total LDL cholesterol and trigliceride levels, and low HDL levels in the plasma) ii) Impaired glucose metabolism (positive glucose tolerance test or definitively high glucose levels in the plasma, and, an increased insulin levels in the plasma). Clinical manifestation of the metabolic syndrome are (i) Arterial hypertension, (ii) Type 2 (non-insulin dependent) diabetes, (iii) Obesity and (iv) Fatty liver. Recent studies suggested that chronic low-grade bacterial infections play a central role in the pathogenesis of these diseases. They may occur alone but more frequently they combine each with other. We selected 60 patients with age 50-60 each them affected by diabetes and high blood lipid levels. They were randomly selected into two groups, 30 patient in each. In addition to their current treatment, which remained unchanged during the study period, the following additional treatments were applied: The control group received a multivitamin every day, while the treated group received full extract of LB in intramuscular injection (0.32 mg), ones every 14 days and a commercially available soft-gel composition of omega 3 FA (1000 mg)+vitamin E (10 mg) every day. This treatment was followed for 3 months. Labor tests and visual field test were performed at the beginning and at the end of this study. Results are summarized in the Table 10

TABLE 10

| | | LB + omega 3 + | |
|---|---|---|---|
| | Control | vitamin E | |
| | (Multivitamin) | (Example II/3 | p |
| Total cholesterol | +8% | −5% | <0.01 |
| HDL cholesterol | −4% | +9% | <0.05 |
| Triglyceride | 0% | −8% | ns |
| Glucose | +3 | −5% | ns |
| CRP | +2% | −8% | <0.01 |
| Blood pressure | +3% | −5% | ns |
| Visual Field | 0% | −10% | <0.05 |
| Defect | | | |

Results: both laboratory tests and visual field test improved in the treated group while worsened or unchanged in the control group. These changes were not significant compared to the baseline, but the improvement of total cholesterol, HDL cholesterol and CRP levels and visual field were statistically significant if were compared to the controls. These findings clearly demonstrated an excellent therapeutic efficacy of this combination of killed LB and omega 3 FA and vitamin E. These results are further supported by the facts that the treatment period was very short for a chronic disease like the metabolic syndrome.

(*) For the above experiments the following commercially available compounds were used:
omega 3: lipid emulsion containing fish oil
LB: cell free extract of killed Lactobacillus in water (Gynevac®, Vakcina Ltd, Sajogalóc, Hungary
capsaicin: Sigma-Aldrich, St. Louis, Mo.,
Endotoxin: Escherichia coli LPS (Sigma-Aldrich, St. Louis, Mo., USA))
control: lipid emulsion: soy oil-containing aqueous emulsion.

### II. Examples for Composition

### Example II/1

### Injection for i.v Use

| | |
|---|---|
| full extract of killed Lactobacillus | 10 mg |
| water for injection | 1.0 ml |

### Example II/2

### Injection for i.v. Use

| | | |
|---|---|---|
| full extract of killed Lactobacillus | 0.5 | mg |
| omega 3 fatty acids | 500.0 | mg |
| α-tocopherol | 10 | mg |
| water for injection | q.s. ad 10 | ml |

These compounds are mixed using the habitual technology for preparing a lipid emulsion, using the following excipients: glycerol, egg lecithin, and sodium hydroxide for pH adjustment.

### Example II/3

### Infusion

Composition

| | | |
|---|---|---|
| cytoplasmic extract of Bifidobacterium | 1 | mg |
| EPA | 5.0 | g |
| DHA | 2.5 | g |
| α-tocopherol | 0.1 | g |
| water for injection | q.s. ad 100 | ml |

These compounds are mixed using the habitual technology for preparing a lipid emulsion, using the following excipients: glycerol, soy lecithin, and sodium hydroxide for ph adjustment

### Example II/4a

### Soft Gel

Composition

| | |
|---|---|
| cytoplasmic extract of Lactobacillus | 50 mg |
| EPA | 500 mg |
| DHA | 250 mg |
| α-tocopherol | 10 mg |
| water for injection | q.s. |

These compounds are mixed using the habitual technology for preparing a lipid emulsion in soft gel, using the following excipients: glycerol, egg lecithin, and sodium hydroxide for pH adjustment.

### Example II/4b

### Infusion

Composition

| | | |
|---|---|---|
| cytoplasmic extract of Lactobacillus | 1.0 | mg |
| cytoplasmic extract of Bifidobacterium | 1.0 | mg |
| EPA | 500 | mg |
| DHA | 250 | mg |
| α-tocopherol | 10 | mg |
| vitamin B1 | 10 | mg |
| water for injection | q.s. ad 100 | ml |

These compounds are mixed using the habitual technology for preparing a lipid emulsion, using the following excipients: glycerol, e lecithin, and sodium hydroxide for pH adjustment

### Example II/5

### Soft Gel

Composition

| | | |
|---|---|---|
| full extract of killed Saccharomyces cerevisie | 50 | mg |
| omega 3 fatty acids | 500 | mg |
| α-tocopherol | 20 | mg |
| vitamin A | 0.000 | IU |

| | |
|---|---|
| water for injection | q.s. |

These compounds are mixed using the habitual technology for preparing a lipid emulsion in soft gel using the following excipients: glycerol, egg lecithin, and sodium hydroxide for pH adjustment

### Example II/6

### Infusion

Composition

| | | |
|---|---|---|
| full extract of killed Lactobacillus | 1.0 | mg |
| DHA | 1000 | mg |
| CoQ10 | 10 | mg |
| α-tocopherol | 10 | mg |
| alpha lipoic acid | 10 | mg |
| water for injectiom | q.s. ad 100 | ml |

These compounds are mixed using the habitual technology for preparing a lipid emulsion, using the following excipients: glycerol, egg lecithin, and sodium hydroxide for pH adjustment

### Example II/7

### Solution

Composition

| | | |
|---|---|---|
| full extract of killed Lactobacillus | 1 | mg |
| EPA | 50 | mg |
| DHA | 25 | mg |
| α-tocopherol | 1 | mg |
| selected DNA/RNA | 1 | mg |
| water for injection | q.s. ad 1 | ml |

The compounds are mixed using the habitual technology for preparing a lipid emulsion, using the following excipients: glycerol, egg lecithin, and sodium hydroxide for ph adjustment

### Example II/8

### Injection

Composition

| | | |
|---|---|---|
| full extract of killed Lactobacillus | 10 | mg |
| full extract of killed Bifidobacterium | 10 | mg |
| EPA | 50 | mg |
| DHA | 25 | mg |
| α-tocopherol | 1 | mg |
| water for injection | q.s. ad 1 | ml |

These compounds are mixed using the habitual technology for preparing a lipid emulsion, using the following excipients: glycerol, egg lecithin, and sodium hydroxide for pH adjustment

### Example II/9

### Injection

Composition

| | | |
|---|---|---|
| full extract of Lactobacillus | 1 | mg |
| full extract of Bifidobacterium | 1 | mg |
| DHA etylester | 200 | mg |
| EPA etylester | 100 | mg |
| α-tocopherol hydrosoluble | 10 | mg |
| water for injection | q.s. ad 10 | ml |

These compounds are mixed using the habitual technology for preparing a lipid emulsion, using the following excipients, glycerol, egg lecithin, and sodium hydroxide for pH adjustment

### Example II/10

### Ointment/Gel

Composition

| | | |
|---|---|---|
| full extract of killed Lactobacillus | 1 | mg |
| full extract of killed Bifidobacterium | 1 | mg |
| DHA | 200 | mg |
| EPA | 20 | mg |
| α-tocopherol | 10 | mg |
| water for injection | q.s. ad 10 | g |

The compounds are mixed using the habitual technology for preparing a lipid emulsion, using the following excipients: water for injection q.s. ad 100 g hydrophilic ointment/gel, glycerol, soy lecithin, and sodium hydroxide for pH adjustment

### Example II/11

### Gel

Composition:

| | | |
|---|---|---|
| full extract of killed Lactobacillus | 10 | mg |
| DHA | 9.0 | g |
| EPA | 1.0 | g |
| α-tocopherol | 0.1 | g |
| CoQ10 | 0.1 | g |
| Gel-forming vehicle* | ad 100.0 | g |

These compounds are mixed using the habitual technology for preparing a gel. (*e.g. U.S. Pat. No. 5,268,112)

### Example II/12

### Soft-Gel Capsule

Composition:

| | |
|---|---|
| full extract of killed Lactobacillus | 10 mg |
| full extract of killed Bifidobacteria | 10 mg |
| DHA | 750 mg |
| EPA | 50 mg |
| α-tocopherol | 10 mg |

These compounds are mixed using the habitual technology for preparing lipid emulsion, using the following excipients: glycerol, soy lecithin, water for injection q.s., and vegetal gelatine for capsules.

### Example II/13

### Solution/Eye Drop

Composition:

| | | |
|---|---|---|
| full extract of killed Lactobacillus | 1 | mg |
| DHA-ethyl-ester | 90 | mg |
| EPA-ethyl-ester | 10 | mg |
| α-tocopherol ethyl-ester | 1 | mg |
| water for injection | q.s. ad 10 | ml |

The compounds are mixed using the habitual technology for preparing a lipid emulsion, using the following excipients: Glycerol, polyvinyl alcohol, and Sodium hydroxide for pH adjustment.

### Example II/15

### Medium for Cell or Tissue Culture

Composition

| | | |
|---|---|---|
| full extract of killed Lactobacillus | 0.5 | mg |
| full extract of killed Bifidobacterium | 0.5 | mg |
| DHA | 250 | mg |
| EPA | 50 | mg |
| α-tocopherol | 10 | mg |
| Dulbecco medium | ad 100 | ml |

The compounds are mixed using the habitual technology for preparing a lipid emulsion, using the following excipients: glycerol, soy lecithin, and sodium hydroxide for pH adjustment.

1-38. (canceled)

39. A composition in the form of a stable lipid emulsion for inhibiting inflammation in a mammal by the inhibition of lipid peroxide generation in the Plasma Membrane Redox System (PMRS) of cells involved in inflammation, the composition comprising:
i) at least one killed probiotic;
ii) at least one omega 3 fatty acid; and
iii) vitamin E; and
additives including water and an emulgator,
wherein a mass ratio of omega 3 fatty acid to water is 100:1 to 100:10000, and wherein a particle size in the lipid emulsion is 0.001 to 100 microns.

40. A composition according to claim 39, wherein the emulgator comprises lecithin, bile, bile acid, Tween® MT polysorbate or polyvinyl alcohol.

41. A composition according to claim 39, further comprising ubiquinone.

42. A composition according to claim 39, comprising a full extract of at least one killed probiotic, where the probiotic is selected from the group consisting of: a cytoplasmic fraction of killed probiotics, nucleotide components of probiotics, and ingredients derived from genetic modification.

43. A composition according to claim 39, wherein the probiotic is selected from the group consisting of: killed Lactobacilli, killed Bifidobacteria, killed yeasts that are derived of Saccharomyces boulardi, Lactobacillus acidophilus, Lactobacillus casei, Lactobacillus plantarum, Lactobacillus reuteri, Lactobacillus rhamnosus, Lactobacillus GG, Lactobacillus bulgaricus, Lactobacillus bifidus, Lactobacillus caucasicus, Lactobacillus brevis, Lactobacillus cellobiosus, Lactobacillus crispatus, Lactobacillus curvatus, Lactobacillus fermentum, Lactobacillus gasseri, Lactobacillus johnsonii, Lactobacillus salivarus, and/or Bifidobacterium animalis subsp. lactis, Bifidobacterium bifidum, Bifidobacterium breve, Bifidobacterium infantis, Bifidobacterium longum, Bifidobacterium adolescentis, Bifidobacterium animalis, Bifidobacterium thermophilum, Bifidobacterium lactis, Lactococcus lactis (formerly known as Streptococcus lactis), Streptococcus thermophilus, Bacillus coagulans, Enterococcus faecalis, Enterococcus faecium, Saccharomyces boulardii, Saccharomyces cerevisiae and Monascus purpurea.

44. A composition according to claim 39, wherein the omega 3 fatty acid is in a natural form, or source of ALA, EPA or DHA or an ester thereof; triglyceride, or phospholipids preferably phospha-tidyl-inositol, phospha-tidyl-choline, phosphatidyl-ethanolamine, phosphatidyl-serine and sphingo-myelin, or and combinations thereof; wherein the vitamin E is a pure α-tocopherol, β-, γ-, and δ-tocopherol, α-, β-, γ-, and δ-tocotrienol or their natural, semi-synthetic and synthetic ester; and wherein the omega 3 fatty acid is in the form of one of its precursors derived from fish-oil, plant-oil or a combination thereof.

45. A composition in the form of an aqueous solution for inhibiting inflammation in a mammal by the inhibition of lipid peroxide generation in the Plasma Membrane Redox System (PMRS) of cells involved in inflammation, the composition comprising:
i) at least one killed probiotic;
ii) a water-soluble salt or ester of at least one omega 3 FA; and
iii) a water-soluble ester of natural semi-synthetic or synthetic vitamin E; and
additives comprising water, vehicles, preservatives and colorants, wherein a mass ratio of Omega 3 fatty acids to vitamin E is from 100:1 to 100; and wherein a mass ratio of Omega 3 fatty acids to probiotics is 100:0.01 to 100.

46. A composition according to claim 39, wherein the composition is designed for: parenteral application such as intravenous, intramuscular, intradermal, intraarticular, intraocular, intralesional, or subcutaneous application; enteral or nasal application; or topical application in the form of eye-drops, gel, spray, ointment, lotion; liposome encapsulated delivery; or use as a soft-gel.

47. A method for preventing, treating or attenuating inflammatory diseases by inhibiting lipid peroxidation, comprising the application of the composition of claim 39 parenterally, in endovenous infusion, in injection, subcutaneous injection, intramuscular injection, intradermal use, intra-lesional use, para-lesional use, intraarticular use, intraocular use, enterally, orally, transrectally, or topically as eye-drops, gel, spray, ointment, lotions or in liposome.

48. A method for preventing, treating or attenuating inflammation and sepsis accompanied with bacterial, viral and fungal infections, and for preventing, treating or attenuating inflammation evoked by use of a vaccine for influenza, hepatitis, BCG, poliomyelitis, Di-Per-Te (diphtheria-pertussis-tetanus), epidemic parotitis, measles or an antiallergic vaccine, the method comprising the application of the composition of claim 39.

49. A method for preventing, treating, or attenuating inflammation of mucous membranes, wherein the inflammation of mucous membranes is due to conjunctivitis, periodotitis, oesophagitis, reflux disease, gastritis, enteritis, colitis, cholecystitis, cystitis, pyelo-nephritis, sinusitis, bronchitis, vaginitis, prostatitis, and or related diseases selected from the group of hepatitis, cirrhosis, nephritis, pleuritis and fibrosis cystica, the method comprising the application of the composition of claim 39.

50. A method for preventing, treating or attenuating an autoimmune inflammation wherein the autoimmune inflammation is rheumatoid arthritis, juvenile (type I) diabetes, Crohn's disease, colitis ulcerosa, psoriasis, lupus erythematous or multiplex sclerosis, the method comprising the application of the composition of claim 39.

51. A method for preventing, treating or attenuating neuropsychiatric disease, wherein the neuropsychiatric disease is schizophrenia, depression, anxiety or panic-disease, the method comprising the application of the composition of claim 39.

52. A method for preventing, treating or attenuating allergenic inflammation, wherein the allergenic inflammation is bronchial asthma, atopic dermatitis, hay-fever, allergic conjunctivitis or allergic rhinitis, and for preventing, treating, or attenuating metabolic syndrome, and for preventing, treating or attenuating proliferative neovascular disease, wherein the neovascular disease is proliferative retinopathy, retinopathy of prematurity (ROP) and malignant tumors, the method comprising the application of the composition of claim 39.

53. A method for preventing, treating or attenuating age-related degenerative disease wherein the age-related degenerative disease is Alzheimer's disease, Parkinson's disease, amyotropic lateral sclerosis, otosclerosis, osteoporosis, osteoarthritis, sarcopenia, hairlessness or age-related skin changes, and for preventing, treating or attenuating inflammation caused by soft tissue damage, wherein the soft tissue damage is postoperative inflammation, sport injuries, extreme sport activities, contusions, buntings, cancer, lung cancer, colon cancer, and lymphomas and chemotherapy associated inflammations and for preventing, treating or attenuating inflammatory eye diseases, wherein the eye disease is uveitis, diabetic retinopathy, age-related macular degeneration, glaucoma or cataract, the method comprising the application of the composition of claim 39.

54. A method for preventing, treating or attenuating arterial hypertension and atherosclerosis, coronary heart disease, cardiac arrhythmia, chronic heart failure, nephrosis syndrome, ischemia-reperfusion, peripheral vascular diseases, acute cerebral ischemia, chronic cerebral ischemia and stroke, the method comprising the application of the composition of claim 39.

55. A method for preventing, treating or attenuating ischemia-reperfusion, acute cerebral ischemia or chronic cerebral ischemia, the method comprising the application of the composition of claim 39.

56. A method for preparing a metabolic adjuvant to gene-transfer natural and semi-synthetic and synthetic DNA and RNA, and for preparing a metabolic adjuvant to culture medium for cell-culture, tissue-culture and bacterial-culture, and for preparing a metabolic adjuvant to support survival of transplanted stem cells and organs, the method comprising the application of the composition of claim 39 to the adjuvants.

57. A method for preparing omega 3 fatty acid and vitamin E enriched cell and tissue extracts, food supplements, foods, and nutrients for human and veterinary application, the method comprising the application of the composition of claim 39 to the omega 3 fatty acid and vitamin E enriched cell and tissue extracts, food supplements, foods, and nutrients for human and veterinary application.
